(19) United States
(12) Patent Application Publication
(10) Pub. No.: US 2022/0257665 A1
(43) Pub. Date: Aug. 18, 2022

(54) APPLICATION OF ANTIARRHYTHMIC AGENTS TO STEM CELL DERIVED CARDIOMYOCYTES AND USES THEREOF

(71) Applicant: Novo Nordisk A/S, Bagsvaerd (DK)

(72) Inventors: Henning Kempf, Vanloese (DK); Salka Elboel Rasmussen, Vaerloese (DK); Diana Mathilde Roepcke, Vedbaek (DK)

(21) Appl. No.: 17/616,283

(22) PCT Filed: Jun. 5, 2020
(86) PCT No.: PCT/EP2020/065703

§ 371 Date: Dec. 3, 2021

(30) Foreign Application Priority Data
Jun. 6, 2019 (EP) 19178657.3

(51) Int. Cl. A61K 35/34; C12N 5/077

(52) U.S. Cl. CPC A61K 35/34; C12N 5/0657; C12N 2513/00; C12N 2501/415; C12N 2506/02

(57) ABSTRACT

The present invention relates to an antiarrhythmic agent for use in a method for the treatment of heart failure by transplantation of stem cell-derived cardiomyocytes, an antiarrhythmic cardiomyocyte cell population, a method for obtaining the antiarrhythmic cardiomyocyte cell population by in vitro exposure of stem cell derived cardiomyocytes to antiarrhythmic agent, and/or medical use of the antiarrhythmic cardiomyocyte cell population in the prevention of arrhythmia and treatment of heart failure. Specifically, the present invention relates to the transplantation of an antiarrhythmic cardiomyocyte cell population or co-administration of antiarrhythmic agent in vivo with transplanted stem cell derived cardiomyocytes.

### TECHNICAL FIELD

The present invention relates generally to the field of stem cells, more specifically to antiarrhythmic cardiomyocyte cell population, a method for obtaining an antiarrhythmic cardiomyocyte cell population, medical use thereof, in prevention or alleviation of arrhythmia caused due to transplantation of stem cell derived cardiomyocytes, in the treatment of heart failure. The present invention also relates to an antiarrythmic agent for use in a method for the treatment of heart failure by the transplantation of stem cell-derived cardiomyocytes.

### BACKGROUND

The heart is one of the least regenerative organs in the body, and as a result, when cardiac injury occurs, cardiomyocytes die and leave behind a scar area that cannot contract. This leads to reduced pumping power, heart failure and increased morbidity and mortality. Heart disease is the leading cause of death worldwide.

Human pluripotent stem cells are able to differentiate into cardiomyocytes and have been investigated for repair of the injured heart, in cases where cardiomyocytes are lost or malfunctioning.

In the few cases, where cardiomyocytes have been injected into the non-human primate heart from the endocardial side, several types of arrhythmia have been detected during the first 4-6 week after engraftment. These arrhythmias are sustained ventricular tachycardia, non-sustained ventricular tachycardia and accelerated idioventricular rhythm.

The reason for these arrhythmias to arise is currently unknown, but it is not surprising, that they occur transiently, because all cardiomyocytes have the ability to contract and beat. Before the transplanted cells have integrated with the host myocardium, they can be expected to beat on their own. Furthermore, the scar area in the myocardium is known to give rise to arrhythmia in patients, especially from the borderzone. Injection of substances into the scar might be arrhythmogenic in itself.

It has been described in monkeys, that the arrhythmias arising after cell injection are of a different type and origin (coming from ectopic foci) than conventional arrhythmias arising after acute myocardial infarctions and heart failure (re-entry mechanism). This means that it is well-defined that cell-transplantation-induced arrhythmia has to be treated, and that the arrhythmia would not have occurred, if cells had not been injected. Therefore, this type of arrhythmia can be considered a new type of condition caused by the cell transplantation, and a specific treatment of this type of arrhythmia does not exist yet.

To regenerate heart muscle tissue in vivo following a heart insult a multitude of different strategies are considered including various types of pluripotent stem cells-derived cardiac lineage cells, e.g. early cardiovascular progenitors, immature beating cardiomyocytes as well as more matured, e.g. heterotypic tissue engineered cardiac constructs. Generally, all approaches for the generation of such cells in vitro result in cardiomyocytes with a relative immature phenotype that resembles fetal-like cells in the first to second trimester of pregnancy regarding their gene expression profile, cell morphology, sarcomere organization, electrophysiological characteristics as well as their resulting contraction force. Notably, multiple of the various strategies were shown to principally yield cells that are capable of engraftment and maturation following transplantation. However with overall limited efficiency. This is particularly due to the limited understanding of the underlying mechanism mediating engraftment and maturation following transplantation, which urges the need for further studies, in particular to identify additional treatments of the transplanted cardiomyocytes to facilitate their development and integration.

It is an object of the present invention to address the problem of arrhythmia caused by engraftment of stem cell-derived cardiomyocytes in a method for the treatment of heart failure. In particular, it is an object of the present invention to facilitate the integration of the transplanted stem cells into the host myocardium, e.g. in order to avoid, prevent and/or alleviate arrhythmia.

### SUMMARY

The aforementioned objects are achieved by the aspects of the present invention. In addition, the present invention may also solve further problems, which will be apparent from the disclosure of the exemplary embodiments.

In the broadest aspect, the present invention relates to in vitro and in vivo approaches for the prevention or alleviation or treatment of arrhythmia caused due to transplantation of stem cell derived cardiomyocytes in a patient and treatment of heart failure.

In one aspect, the present invention relates to a method for obtaining antiarrhythmic cardiomyocyte cell population comprising the step of culturing stem cell derived cardiomyocytes in a medium comprising one or more anti-arrhythmic agents.

In one aspect, the present invention relates to antiarrhythmic cardiomyocyte cell population for use as a medicament.

In one aspect, the present invention relates to an antiarrhythmic cardiomyocyte cell population for use in the treatment of heart failure.

In one aspect, the present invention relates to an antiarrhythmic cardiomyocyte cell population for use in the prevention or alleviation of arrhythmia caused due to transplantation of stem cell derived cardiomyocytes.

In one aspect, the present invention relates to a kit comprising one or more antiarrhythmic agent and stem cell-derived cardiomyocytes.

In another aspect, the present invention relates to an antiarrhythmic agent for use in a method for the treatment of heart failure by transplantation of stem cell-derived cardiomyocytes.

A further aspect of the present invention relates to a composition comprising stem cell derived cardiomyocytes, one or more antiarrhythmic agents and optionally a biomaterial for use in a method for the treatment of heart failure.

Without being bound by any theory, the present inventors believe that the arrhythmia observed in a host having transplanted stem cell-derived cardiomyocytes is a symptom caused by the stem cells not yet being fully integrated into the myocardium. Now the present inventors have shown approaches of preventing or alleviating arrhythmia and/or treating heart failure. One of the approaches is to obtain antiarrhythmic cardiomyocyte cell population by contacting stem cell-derived cardiomyocytes with an antiarrhythmic agent in vitro that changes the regulation of gene expression on key genes which are associated with the cardiomyocytes being able to connect and contract in synchrony. This is believed to facilitate the integration of the anti arrhythmic cardiomyocyte cell population into the host myocardium after transplantation thereby preventing or alleviating the aforementioned antiarrhythmic effects and provide a treatment of heart failure by the suppression of the stem cell-derived cardiomyocytes' ability to contract and beat independently. Another approach is to co-administer one or more anti arrhythmic agent in vivo during or after transplantation of stem cell derived cardiomyocytes into a patient.

### BRIEF DESCRIPTION OF DRAWINGS

FIG. 1 shows gene expression pattern of CACNA1G after 5 days exposure to 0.1 μM and 1 μM amiodarone on stem cell-derived cardiomyocytes (day 23 after differentiation induction). Negative and positive control refer to immature (day 9) and mature (day 42) cardiomyocytes, respectively.

FIG. 2 shows gene expression pattern of GJA5 after 5 days exposure to 0.1 μM and 1 μM amiodarone on stem cell-derived cardiomyocytes (day 23 after differentiation induction). Negative and positive control refer to immature (day 9) and mature (day 42) cardiomyocytes, respectively.

FIG. 3 shows gene expression pattern of NPPA after 5 days exposure to 0.1 μM and 1 μM amiodarone on stem cell-derived cardiomyocytes (day 23 after differentiation induction). Negative and positive control refer to immature (day 9) and mature (day 42) cardiomyocytes, respectively.

FIG. 4 shows gene expression pattern of NPPB after 5 days exposure to 0.1 μM and 1 μM amiodarone on stem cell-derived cardiomyocytes (day 23 after differentiation induction). Negative and positive control refer to immature (day 9) and mature (day 42) cardiomyocytes, respectively.

FIG. 5 shows gene expression pattern of NKX2-5 after 5 days exposure to 0.1 μM and 1 μM amiodarone on stem cell-derived cardiomyocytes (day 23 after differentiation induction). Negative control refers to immature (day 9) cardiomyocytes.

FIG. 6 shows gene expression pattern of TNNT2 after 5 days exposure to 0.1 μM and 1 μM amiodarone on stem cell-derived cardiomyocytes (day 23 after differentiation induction). Negative control refers to immature (day 9) cardiomyocytes.

FIG. 7 shows gene expression pattern of ACTA2 after 5 days exposure to 0.1 μM and 1 μM amiodarone on stem cell-derived cardiomyocytes (day 23 after differentiation induction). Negative control refers to immature (day 9) cardiomyocytes.

FIG. 8 shows gene expression pattern of SCNSA after 5 days exposure to 0.1 μM and 1 μM amiodarone on stem cell-derived cardiomyocytes (day 23 after differentiation induction). Negative control refers to immature (day 9) cardiomyocytes.

FIG. 9 shows gene expression pattern of NPPA after 5 days exposure to 1 μM, 10 μM and 100 μM lidocaine on stem cell-derived cardiomyocytes (day 23 after differentiation induction). Negative control refers to immature (day 9) cardiomyocytes.

FIG. 10 shows gene expression pattern of NPPB after 5 days exposure to 1 μM, 10 μM and 100 μM lidocaine on stem cell-derived cardiomyocytes (day 23 after differentiation induction). Negative control refers to immature (day 9) cardiomyocytes.

FIG. 11 shows gene expression pattern of NKX2-5 after 5 days exposure to 1 μM, 10 μM and 100 μM lidocaine on stem cell-derived cardiomyocytes (day 23 after differentiation induction). Negative control refers to immature (day 9) cardiomyocytes.

FIG. 12 shows gene expression pattern of TNNT2 after 5 days exposure to 1 μM, 10 μM and 100 μM lidocaine on stem cell-derived cardiomyocytes (day 23 after differentiation induction). Negative control refers to immature (day 9) cardiomyocytes.

FIG. 13 shows gene expression pattern of SCN5A after 5 days exposure to 0.1 μM, 1 μM amiodarone on stem cell-derived cardiomyocytes (day 23 after differentiation induction). Negative control refers to immature (day 9) cardiomyocytes.

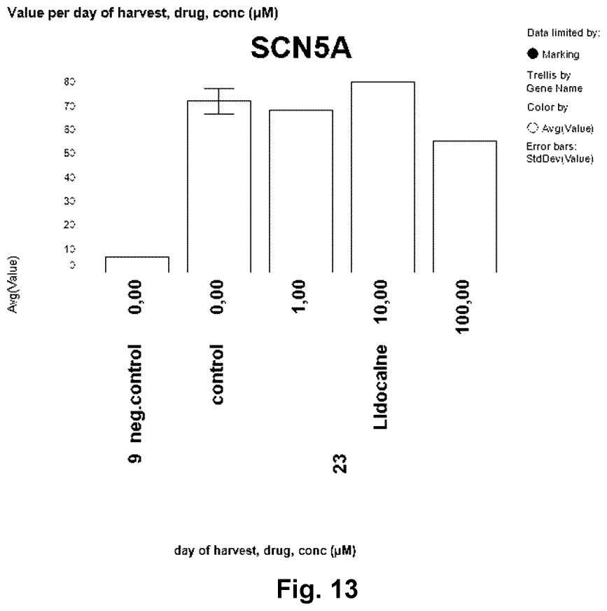

FIG. 14 shows the gene expression for CACNA1G, GJA5, NPPA and NPPB of 21 day old stem-cell derived cardiomyocytes after 5-days exposure to 10 μM amiodarone followed by a 2 day recovery period in the absence of the drug. Respective untreated cardiomyocytes are shown as controls. The cardiomyocytes were maintained as three-dimensional suspension clusters of about 150 μm-300 μm diameter size throughout the experiment.

FIG. 15 shows coefficient of variation (CV) of the beat-to-beat variability of stem cell derived cardiomyocytes after exposure with an anti-arrhythmic drug at indicated concentrations at baseline (left) and following stimulation with 200 nM moxifloxacine that is a proarrhythmic initiator (right panel). Bar graphs represent mean+standard error of mean. N=12 and N=18 for compound treated conditions and control, respectively. The asterisk indicates statistical significance (p<0.05) based on a Kruskal-Wallis test comparing all compounds to control treatment.

FIG. 16 shows coefficient of variation (CV) of the beat-to-beat variability of stem cell derived cardiomyocytes after exposure to combinations of anti-arrhythmic agents applied at the following concentrations: 1 μM sotalol, 0.1 μM amiodarone, 0.1 μM metoprolol 1 μM mexiletine, 1 μM propranolol and compared to single agents. All conditions were measured following stimulation with 200 nM moxifloxacine. Bar graphs represent mean+standard error of mean. N=70 and N=12 for single compound treated conditions and compound combinations, respectively. The asterisk indicate statistical significance (p<0.05) based on a Kruskal-Wallis test comparing each group of compound treatments to single compound controls.

### DESCRIPTION

Unless otherwise stated, all technical and scientific terms used herein have the same meanings as commonly understood by one of ordinary skill in the art to which this invention belongs. The practice of the present invention employs, unless otherwise indicated, conventional methods of chemistry, biochemistry, biophysics, molecular biology, cell biology, genetics, immunology and pharmacology, known to those skilled in the art.

It is noted that all headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Throughout this application the terms “method” and “protocol” are used interchangeably.

Throughout this application the terms “culturing”, “contacting” and “exposing” are used interchangeably.

Throughout this application the terms “human subject”, “patient” and “host” are used interchangeably.

As used herein, “a” or “an” or “the” can mean one or more than one. Unless otherwise indicated in the specification, terms presented in singular form also include the plural situation.

Also as used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

The term “about,” as used herein when referring to a measurable value such as an amount of cells, a compound or an agent of this invention, dose, temperature, and the like, is meant to encompass variations of 5%, 1%, 0.5%, or even 0.1% of the specified amount.

As used herein, the term “day” in reference to the protocols refers to a specific time for carrying out certain steps. In general and unless otherwise stated “day 0” refers to the initiation of the protocol, this be by plating the stem cells or transferring the stem cells to an incubator or contacting the stem cells in their current cell culture medium with a compound prior to transfer of the stem cells. Typically, the initiation of the protocol will be by transferring undifferentiated stem cells to a different cell culture medium and/or container such as by plating or incubating, and/or with the first contacting of the undifferentiated stem cells with a compound that affects the undifferentiated stem cells in such a way that a differentiation process is initiated.

When referring to “day x”, such as day 1, day 2 etc., it is relative to the initiation of the protocol at day 0. One of ordinary skill in the art will recognize that unless otherwise specified the exact time of the day for carrying out the step may vary. Accordingly, “day x” is meant to encompass a time span such as of +/−10 hours, +/−8 hours, +/−6 hours, +/−4 hours, +/−2 hours, or +/−1 hours. Alternatively, the duration or time for carrying out steps of the method according to the present invention is described in “hours”.

Hereinafter, the methods according to the present invention are described in more detail by non-limiting embodiments and examples.

As used herein, the term “arrhythmia” means a condition in which the heart beats with an irregular or abnormal rhythm. In macaques, it has been shown that the specific cell-induced arrhythmias are non-sustained ventricular tachycardia, sustained ventricular tachycardia and sustained accelerated idioventricular rhythm.

Accordingly, one embodiment the treatment of arrhythmia is of non-sustained ventricular tachycardia, sustained ventricular tachycardia and/or sustained accelerated idioventricular rhythm.

As used herein, the term “antiarrhythmic agent” or “antiarrhythmic drugs” or “antiarrhythmic compounds” or “antiarrhythmics” means one or more pharmaceuticals divided into different drug classes (the Vaughan Williams classes), depending on mode of action. Class I drugs primarily block the sodium channels, class II drugs block the beta receptors, class III drugs block the potassium channels, while class IV drugs affect the calcium channels. It is important to note, that one drug class also can have an effect on more than one ion channel type, but is categorized by its main function.

In one embodiment, the antiarrhythmic agent is selected from the list of antiarrhythmic agents of class I, class II, class III, class IV, and class V. In a further embodiment, the antiarrhythmic agent is selected from the list of antiarrhythmic agents of class I, class II, and class III. In an embodiment, the antiarrhythmic agent is a class III antiarrhythmic agent.

As used herein, the term “antiarrhythmic cardiomyocyte cell population” is to be understood as cardiomyocytes obtained by the method of the present invention having modified properties resulting in reduction of beat-to-beat variability and/or reduction in their susceptibility to arrhythmias and/or other arrhythmic like events.

As used herein, the term “biomaterial” refers to any chemical substance of synthetic or natural materials with the purpose of interacting with the cell product. Such biomaterials comprise but are not limited to the following groups of materials, natural and/or synthetic polymeric materials comprising alginate, chitosan, cellulose, agarose, gelatine, hyaluronic acid, silk fibroin, fibrin and/or collagen, poly-urethane, poly-vinyl alcohol, poly-hydroxy esters, poly-propylene fumarate as well as other synthetic, biodegradable and/or stimuli-sensitive hydrogels, bioactive glasses.

The terms “cardiac muscle cells”, “cardiomyocytes”, “myocardiocytes” and “cardiac myocytes” may be used interchangeably and refer to the muscle cells that make up the cardiac muscle (heart muscle). Each myocardial cell contains myofibrils, which are specialized organelles consisting of long chains of sarcomeres, the fundamental contractile units of muscle cells.

As used herein, the term “heart failure” is meant an inability of the heart to keep up with the demands on it and, specifically, failure of the heart to pump blood with normal efficiency. When this occurs, the heart is unable to provide adequate blood flow to other organs such as the brain, liver and kidneys. Heart failure may be due to failure of the right or left or both ventricles. Specifically, the heart failure may be a myocardial infarction, commonly known as a heart attack, which occurs when blood flow decreases or stops to a part of the heart, causing damage to the heart muscle. If impaired blood flow to the heart lasts long enough, it triggers an ischemic cascade; the heart cells in the territory of the blocked coronary artery die (infarction), chiefly through necrosis, and do not grow back.

By “stem cell” is to be understood as an undifferentiated cell having differentiation potency and proliferative capacity (particularly self-renewal competence), but maintaining differentiation potency. The stem cell includes subpopulations such as pluripotent stem cell, multipotent stem cell, unipotent stem cell and the like according to the differentiation potency. Pluripotent stem cell refers to a stem cell capable of being cultured in vitro and having a potency to differentiate into any cell lineage belonging to three germ layers (ectoderm, mesoderm, endoderm) and/or extraembryonic tissue (pluripotency). The multipotent stem cell means a stem cell having a potency to differentiate into plural types of tissues or cells, though not all kinds. The unipotent stem cell means a stem cell having a potency to differentiate into a particular tissue or cell. A pluripotent stem cell can be induced from fertilized egg, clone embryo, germ stem cell, stem cell in a tissue, somatic cell and the like. Examples of the pluripotent stem cell include embryonic stem cell (ES cell), EG cell (embryonic germ cell), induced pluripotent stem cell (iPS cell) and the like. Muse cell (Multi-lineage differentiating stress enduring cell) obtained from mesenchymal stem cell (MSC), and GS cell produced from reproductive cell (e.g., testis) are also encompassed in the pluripotent stem cell. Induced pluripotent stem cells (also known as iPS cells or iPSCs) are a type of pluripotent stem cell that can be generated directly from adult cells. By the introduction of products of specific sets of pluripotency-associated genes adult cells can be converted into pluripotent stem cells. Embryonic stem cells can be produced by culturing an inner cell mass obtained without the destruction of the embryo. Embryonic stem cells are available from given organizations and are also commercially available.

As used herein, the term “stem cell-derived cardiomyocytes” is to be understood as cardiomyocytes at various stages of development, which have been derived through an in vitro protocol to obtain a non-native stem cell product resembling the muscle cells of the human heart. In one embodiment, the stem cell-derived cardiomyocytes are derived from human pluripotent stem cells, such as human embryonic stem cells. In one embodiment, the stem cell-derived cardiomyocytes are derived from induced pluripotent stem cells. In one embodiment, the stem cell-derived cardiomyocytes are derived from other sources such as transdifferentiation of somatic cells to cardiomyocytes (Masaki leda et al, Direct Reprogramming of Fibroblasts into Functional Cardiomyocytes by Defined Factors, Volume 142, Issue 3, P375-386, Aug. 6, 2010). A person skilled in the art will be able to provide stem cell-derived cardiomyocytes. One available method as used in the present invention is described (in “Kempf H et al Bulk cell density and Wnt/TGFbeta signalling regulate mesendodermal patterning of human pluripotent stem cells. Nat Commun. 2016; 7:13602”.) In one embodiment, stem cell-derived cardiomyocytes include precursors or progenitors thereof. The stem cell derived cardiomyocyte population is typically characterized by the expression of at least 3 of the markers selected from NKX2.5, TNNT2, ACTN2, MYH6 and/or MYH7, MYL2 and/or MYL7, TNNI1 and/or TNNI3. Depending on the maturation state of the stem cell derived cardiomyocytes, the stem cell derived cardiomyocytes or progenitors or precursors thereof can comprise of cells expressing ISL1, GATA4, MEF2C, SSEA-1, PDGFRA, MESP1 and/or a combination thereof.

As referred to herein, the terms “transplantation” and “engraftment” may be used interchangeably and refer to the process of taking viable stem cell-derived cardiomyocytes or antiarrhythmic cardiomyocyte cell population obtained according to the method of the present invention and implanting them into or in the vicinity of the heart of a human subject or a patient.

As referred to herein, the terms “transplant” and “graft” refer to the stem cell-derived cardiomyocytes or antiarrhythmic cardiomyocyte cell population obtained according to the method of the present invention being transferred into a human subject or patient via the aforementioned procedure.

An aspect of the present invention relates to an antiarrhythmic agent for use in a method for the treatment of heart failure by transplantation of stem cell-derived cardiomyocytes.

In one embodiment according to this aspect, stem cell derived cardiomyocytes are transplanted into a patient.

In one embodiment according to this aspect, one or more antiarrhythmic agent are for co-administration with the transplantation of stem cell derived cardiomyocytes in vivo.

In one embodiment according to this aspect, one or more antiarrhythmic agent are co-administered during the transplantation of the stem cell derived cardiomyocytes.

In one embodiment according to this aspect, one or more antiarrhythmic agent are co-administered after the transplantation of the stem cell derived cardiomyocytes.

This co-administration of antiarrhythmic agent with the transplantation of stem cell derived cardiomyocytes can be further improved by combining the cells and the agent with a biomaterial such as a hydrogel to enable prolonged and locally restricted release of the agent at its target site (Jianyu Li and David J. Mooney, Designing hydrogels for controlled drug delivery, Nat Rev Mater. 2016 December; 1(12): 16071. Published online 2016 Oct. 18), e.g. by incorporation of the arrhythmic agent in biodegradable hydrogel particles (Radhika Narayanaswamy, Vladimir P Torchilin, Hydrogels and Their Applications in Targeted Drug Delivery, Molecules. 2019 February; 24(3): 603. Published online 2019 Feb. 8).

In one aspect the present invention relates to a method for obtaining antiarrhythmic cardiomyocyte cell population comprising the step of culturing stem cell derived cardiomyocytes in a medium comprising one or more anti-arrhythmic agents.

In one embodiment, the present invention relates to a method for obtaining antiarrhythmic cardiomyocyte cell population comprising the step of in vitro culturing stem cell derived cardiomyocytes or precursors or progenitors thereof in a medium comprising one or more anti-arrhythmic agents.

In one embodiment, the stem cell derived cardiomyocytes are cultured in a medium comprising an antiarrhythmic agent for less than 24 hours, at least 24 hours, from 24-48 hours, at least 48 hours, from 48-72 hours, at least 72 hours, from 72-96 hours, at least 96 hours.

In one embodiment, the stem cell derived cardiomyocytes are cultured with from 1 nM-100 nM, about 1 nM, about 10 nM, about 20 nM, about 40 nM, about 60 nM, about 80 nM, about 100 nM or from 0.1-100 μM, 0.5 μM, 1 μM, 5 μM, 10 μM, 100 μM of class I, class II and/or class III antiarrhythmic agents.

In one embodiment according to this aspect, an antiarrythmic cardiomyocyte cell population is transplanted into a patient.

Another aspect of the present invention relates to an antiarrythmic cardiomyocyte cell population for use as a medicament. In one embodiment according to this aspect, present invention relates to an antiarrythmic cardiomyocyte cell population for use in the method for treatment of heart failure. In one embodiment according to this aspect, present invention relates to an antiarrythmic cardiomyocyte cell population for use in the prevention or alleviation of arrhythmia caused due to transplantation of stem cell derived cardiomyocytes. In one embodiment, the present invention relates to prevention or alleviation of proarrhythmia.

An advantageous benefit of transplantation of antiarrhythmic cardiomyocyte cell population in a patient is that it reduces the typical risk of proarrhythmic effects (e.g. bradycardia, A-V block, prolonged QT intervals) from anti-arrhythmic agent (D P Zipes, Proarrhythmic Effects of Antiarrhythmic Drugs, 1987 Apr. 30; 59(11):26E-31E) and other side effects, including but not limited to interstitial pulmonary fibrosis, hypo- and hyperthyroidism, liver toxicity, hypotension, tremor, dizziness, slight fever, photosensitivity, neuropathy, muscle weakness (Thomas W. Nygaard et al, Adverse Reactions to Antiarrhythmic Drugs During Therapy for Ventricular Arrhythmias, JAMA. 1986; 256(1):55-57); Janice B. Schwartz et al, Adverse Effects of Antiarrhythmic Drugs, Drugs volume 21, pages 23-45(1981).

An advantage of antiarrhythmic cardiomyocyte cell population is that it is likely to show superior engraftment into the host myocardium compared to stem cell derived cardiomyocytes as the reduced pro-arrhythmic potential enables a synchronous beating behaviour, which facilitates fast and/or correct integration of the cells and enables a faster maturation process.

Additional advantage of the present invention is that in vitro exposure of the stem-cell derived cardiomyocytes to the anti-arrhythmic agents allows exposure at higher concentration levels of at least 10, 100, 1000, or 10000-fold above typical plasma concentrations in vivo i.e. 2-6 μg/ml for Lidocaine, 0.6-1.7 μg/ml for Mexiletine, 2.1-300 ng/ml for propranolol (Plasma concentrations of propranolol and 4-hydroxypropranolol during chronic oral propranolol therapy, Br J Clin Pharmacol. 1979 August; 8(2): 163-167), 100-140 ng/ml for metoprolol (Plasma levels and effects of metoprolol on blood pressure and heart rate in hypertensive patients after an acute dose and between two doses during long-term treatment, Clinical pharmacology and therapeutics, first published: April 1975) 0.5-2.5 μg/ml for amiodarone, 1-3 μg/ml for Sotalol) thereby increasing the likelihood of success for obtaining a antiarrhythmic cardiomyocyte cell population compared to in vivo treatment.

Overall, the antiarrhythmic cardiomyocyte cell population has an increased likelihood of successful integrating into the host myocardium compared to typical stem-cell derived cardiomyocytes, thereby improving the outcome of the cell transplantation by increasing the overall pump function of the recipients heart.

In a further embodiment, the antiarrhythmic cardiomyocyte cell population has at least 50% reduction in coefficient of variation (CV) or beat to beat variability when compared to stem cell derived cardiomyocytes.

Antiarrhythmic drugs such as amiodarone and others can be administered as a solution, tablet, hydrogel-encapsulation, etc. and by various routes of administration such as intravenous, oral, intrapericardial, etc. This has been shown in patients (Garcia J R et al., Minimally invasive delivery of hydrogel-encapsulated amiodarone to the epicardium reduces atrial fibrillation). In one embodiment the class III antiarrhythmic agent is sotalol. As used herein, “sotalol” refer CAS number 3930-20-9 with formula C12H20N203S. In one embodiment, concentration of sotalol is 100 nM.

In one embodiment of the present invention, antiarrhythmic cardiomyocyte cell population is obtained in vitro by culturing stem cell derived cardiomyocytes in a medium comprising one or more anti-arrhythmic agents.

One embodiment of the present invention relates to antiarrhythmic agent for use in a method for the treatment of heart failure by transplantation of stem cell-derived cardiomyocytes.

In one embodiment the class III antiarrhythmic agent is amiodarone. As used herein, “amiodarone” refers CAS number 1951-25-3 with formula C25H29I2NO3. Amiodarone especially is found to increase the expression of gap junctions (GJA5), which are cellular membrane constructs that enable cardiomyocytes to connect and contract in synchrony. This is a pivotal part of how cardiomyocytes work together and ensure normal propagation of the electromechanical impulses that ensure proper contraction of the heart. This finding supports that amiodarone increases engraftment and integration of the stem cell derived cardiomyocytes into host tissue and help in ensuring synchronized contraction of the cardiomyocytes.

Furthermore, amiodarone is found to increase CACNA1G expression. Calcium handling is a very important part of contractility of the cells and generation of action potentials. Therefore, this finding supports the stabilizing effect of the drug on rhythm and contraction of the cells.

Amiodarone is also found to suppress ANP and BNP expression. This indicates a possible suppression of a hypertrophic response or simply be an indicator of better functioning cardiomyocytes. ANP and BNP are known to rise when heart failure worsens, so if ANP and BNP are low, this is a sign of more well-functioning cardiomyocytes and heart. The findings support that amiodarone improves functionality of the stem cell-derived cardiomyocytes or antiarrythmic cardiomyocyte cell population. In one embodiment, concentration of amiadarone is 10 nM.

In another embodiment, the antiarrhythmic agent is a class I antiarrhythmic agent. In one embodiment the class I antiarrhythmic agent is lidocaine. As used herein, “lidocaine” refers to CAS number 137-58-6 with chemical formula C14H22N2O. In one embodiment, concentration of lidocaine is 100 nM. In one embodiment, the class I antiarrhythmic agent is Mexiletine. As used herein, “mexiletine” refers to CAS number 31828-71-4 with formula C11H17NO. In one embodiment, concentration of Mexiletine is 100 nM.

In an alternative embodiment, the antiarrhythmic agent is a class II antiarrhythmic agent. In one embodiment, the class II antiarrhythmic agent is metoprolol. As used herein, “metoprolol” refers to CAS number 51384-51-1 with chemical formula C158H25NO3. In one embodiment, concentration of metoprolol is 10 nM.

In one embodiment, the class II antiarrhythmic agent is propranolol. As used herein, “propranolol” refers to CAS number 525-66-6 with formula C16H21NO2. In one embodiment, concentration of propranolol is 100 nM.

In one embodiment the antiarrhythmic agent is construed as only a single compound. In one embodiment, antiarrhythmic agent is a formulation. In one embodiment, antiarrhythmic agent is a combination of one or more antiarrhythmic agent(s) selected from the same or different classes.

As described herein the treatment of a patient undergoing transplantation of stem cell-derived cardiomyocytes may be by administration of the one or more antiarrhythmic agent(s) by any suitable means. The antiarrhythmic agent may be formulated in any suitable way for administration, such as but not limited administration by intravenous injection with an injection device or by ingestion as a tablet. Injection device refers to a medical grade system intended for the delivery of the cellular product in the respective formulation to the recipient. Injection device is preferentially suitable for pericardial, epicardial and/or intracardial delivery. Injection device can comprise but is not limited to needle-tipped syringes, needle-free syringes, injection catheter systems suitable for delivery in the proximity of the myocardial infarct area. Injection device comprises but is not limited to devices intended for intracoronary, endocardial and/or epicardia injection.

When the antiarrhythmic agent comprises more than one antiarrhythmic compound it may or may not be co-formulated and it may or may not be administered together or separate and/or in different dosage regimes and/or at different time intervals.

In a preferred embodiment, the antiarrhythmic agent is a combination of at least two classes of antiarrhythmic agents. In an embodiment the antiarrhythmic agent comprises a class III antiarrhythmic agent and a class I antiarrhythmic agent. In one embodiment, the antiarrhythmic agent comprises amiodarone and a class I antiarrhythmic agent. In one embodiment, the antiarrhythmic agent comprises sotalol and a class I antiarrhythmic agent. In one embodiment the class I antiarrhythmic agent is lidocaine. In one embodiment the class I antiarrhythmic agent is mexiletine.

In one embodiment, the antiarrhythmic agent comprises amiodarone and lidocaine.

In one embodiment, the antiarrhythmic agent comprises amiodarone and mexiletine.

In one embodiment, the antiarrhythmic agent comprises sotalol and lidocaine.

In one embodiment, the antiarrhythmic agent comprises sotalol and mexiletine.

In one embodiment, antiarrhythmic agent comprises 1 μM sotalol and 1 μM mexiletine.

In an embodiment the antiarrhythmic agent comprises a class III antiarrhythmic agent and a class II antiarrhythmic agent. In one embodiment, the antiarrhythmic agent comprises amiodarone and a class II antiarrhythmic agent. In one embodiment, the antiarrhythmic agent comprises sotalol and a class II antiarrhythmic agent. In one embodiment the class II antiarrhythmic agent is metoprolol. In one embodiment the class II antiarrhythmic agent is propranolol.

In one embodiment, the antiarrhythmic agent comprises amiodarone and metoprolol.

In one embodiment, the antiarrhythmic agent comprises amiodarone and propranolol.

In one embodiment, antiarrhythmic agent comprises 0.1 μM amiodarone and 1 μM propranolol.

In one embodiment, the antiarrhythmic agent comprises sotalol and metoprolol.

In one embodiment, antiarrhythmic agent comprises 0.1 μM metoprolol and 1 μM sotalol.

In one embodiment, the antiarrhythmic agent comprises sotalol and propranolol.

In an embodiment the antiarrhythmic agent comprises a class I antiarrhythmic agent and a class II antiarrhythmic agent.

In one embodiment, the antiarrhythmic agent comprises lidocaine and metoprolol.

In one embodiment, the antiarrhythmic agent comprises lidocaine and propranolol.

In one embodiment, the antiarrhythmic agent comprises mexiletine and metoprolol.

In one embodiment, antiarrhythmic agent comprises 0.1 μM metoprolol and 1 μM mexiletine. In one embodiment, the antiarrhythmic agent comprises mexiletine and propranolol.

In an embodiment the antiarrhythmic agent comprises a combination of two same classes of antiarrhythmic agent. In an embodiment the antiarrhythmic agent comprises two agents from class I. In one embodiment, the antiarrhythmic agent comprises lidocaine and mexiletine.

In an embodiment the antiarrhythmic agent comprises two agents from class II. In one embodiment, the antiarrhythmic agent comprises metoprolol and propranolol.

In an embodiment the antiarrhythmic agent comprises two agents from class III. In one embodiment, the antiarrhythmic agent comprises amiodarone and sotalol.

In one embodiment, the antiarrhythmic agent comprises a combination of three classes of antiarrhythmic agent. In one embodiment, the antiarrhythmic agent comprises a combination of class I, class and class III antiarrhythmic agent.

The effect of the drugs is enhanced when two or more drugs are combined, which only supports the finding that the drugs have a relevant effect on beating/frequency, and that a combination of drugs is more effective than single drug treatment.

The present inventors found that overall, all the drugs show an effect on beating when concentrations are increased, i.e. for low concentrations the cells are beating and slow their beating frequency, at medium concentrations the cells stop beating, and at high concentrations the drugs are known to be toxic so it is expected that cells die, if concentrations get too high. This confirms that the drugs have a relevant anti-arrhythmic effect with a dose-dependent effect on beating frequency. This result therefore confirms that beating and rhythm are improved by the drugs, making arrhythmia less likely to arise.

Another aspect of the present invention relates to an antiarrhythmic agent for use in a method for the treatment or prevention of arrhythmia caused by the transplantation of stem cell-derived cardiomyocytes. In a specific embodiment, the arrhythmia is caused by a method for the treatment of heart failure by transplantation of stem cell-derived cardiomyocytes.

In one embodiment, the method of treatment is for obtaining a high probability of successful transplantation outcome of the transplanted stem cell-derived cardiomyocytes or transplanted anti-arrhythmic cardiomyocyte cell population.

In another embodiment the method of treatment is for facilitating a safer engraftment of the transplanted stem cell-derived cardiomyocytes or transplanted anti-arrhythmic cardiomyocyte cell population into a host myocardium.

In a further embodiment thereof, the method of treatment is for improving beating and/or rhythm of the transplanted stem cell-derived cardiomyocytes or transplanted anti-arrhythmic cardiomyocyte cell population. In a further embodiment thereof, the method is for reducing the variation in beating and/or rhythm of the transplanted stem cell-derived cardiomyocytes or transplanted anti-arrhythmic cardiomyocyte cell population.

In another aspect of the present invention, the antiarrhythmic agent is for use in a method for the prevention of graft rejection following the transplantation of antiarrythmic cardiomyocyte cell population due to an altered gene expression in the engrafted cells following the exposure to the antiarrhythmic agent in vitro. It follows that the present inventors have shown that the antiarrhythmic agent directly affects the stem cell-derived cardiomyocytes.

Another aspect of the present invention relates to a composition comprising stem cell derived cardiomyocytes, one or more antiarrhythmic agent, and optionally a biomaterial for use in a method for the treatment of heart failure by transplantation of stem cell-derived cardiomyocytes. In one embodiment, stem cell derived cardiomyocytes are single cells, cell clusters or cell patches. In one embodiment of the composition, the class I antiarrhythmic agent is amiodarone, and the class III antiarrhythmic agent is lidocaine.

Another aspect of the present invention relates to a kit comprising an antiarrhythmic agent and stem cell-derived cardiomyocytes. In one embodiment, the kit is for use in a method for the treatment of heart failure, preferably by the transplantation of the stem cell-derived cardiomyocytes. In one embodiment the antiarrhythmic agent is selected from the list of class I, class II, class III, class IV, and class V antiarrhythmic agents, or a combination thereof. In a preferred embodiment, the antiarrhythmic agent is selected from the list of class I, class II, and class III antiarrhythmic agents, or a combination thereof. In a further embodiment, the class I antiarrhythmic agent is lidocaine, the class antiarrhythmic agent is metoprolol, and/or the class III antiarrhythmic agent is amiodarone, or a combination thereof. In a preferred embodiment, the kit comprises amiodarone and lidocaine.

Another aspect of the present invention relates to a method for obtaining antiarrythmic cardiomyocyte cell population with a high probability of successful transplantation outcome, comprising a step of regulating the expression of a gene selected from the list of GJA5, CACNA1G, NPPA and/or NPPB.

In a preferred embodiment, the step of regulating the expression of the gene is carried out by contacting the stem cell-derived cardiomyocytes with an antiarrhythmic drug in vitro.

In one embodiment, the gene CACNA1G is upregulated more than about 1.5 times, such as more that about 2 times, In one embodiment, the gene GJA5 is upregulated more than about 2 times. In one embodiment, the gene NPPA is downregulated more than about 2 times. In one embodiment, the gene PPB is downregulated more than about 2 times, such as more than about 3 times, preferably more than about 4 times. In one embodiment, the regulation of the gene expression is in vitro. As used in this context by “in vitro” is meant a cell population outside the human body, e.g. contained in a suitable vessel.

It follows that a unique cell population i.e. antiarrythmic cardiomyocyte cell population is obtained according to the method of the aforementioned aspect. Accordingly, another aspect of the present invention relates to a antiarrythmic cardiomyocyte cell population, wherein at least 40%, 50%, 60%, 70%, 80, 90%, 95%, or 99% of the cardiomyocytes have regulated gene expression, wherein CACNA1G is upregulated by at least about 1.5 times, and/or GJA5 is upregulated by at least about 2 times, and/or NPPA is downregulated by at least about 2 times, and/or NPPB is downregulated by at least about 2 times. In one embodiment, the antiarrythmic cardiomyocyte cell population has been obtained in vitro.

In another embodiment the antiarrythmic cardiomyocyte cell population is used in in vitro assays including but not limited to drug screening, toxicity testing and/or disease modelling.

Another aspect of the present invention relates to a method for the treatment of heart failure, comprising the steps of: a) obtaining in vitro stem cell-derived cardiomyocytes, b) transplanting the stem cell-derived cardiomyocytes into a patient, and c) co-administering an antiarrhythmic agent to the patient in vivo during or after transplantation. In a preferred embodiment, the antiarrhythmic agent in step c) comprises amiodarone and lidocaine.

In one embodiment the method comprises the step of contacting in vitro the stem cell-derived cardiomyocytes with an antiarrhythmic agent to obtain antiarrhythmic cardiomyocyte cell population that is transplanted to the patient. In a preferred embodiment, the method comprises the step of contacting in vitro the stem cell-derived with amiodarone and lidocaine.

### PARTICULAR EMBODIMENTS

The aspects of the present invention are now further described by the following non-limiting embodiments:
1. A method for obtaining antiarrhythmic cardiomyocyte cell population comprising the step of culturing stem cell derived cardiomyocytes in a medium comprising one or more anti-arrhythmic agents.
2. The method according to embodiment 1, wherein the stem cell derived cardiomyocytes are cultured in a medium for less than 24 hours.
3. The method according to embodiment 1, wherein the stem cell derived cardiomyocytes are cultured with one or more anti-arrhythmic for at least 24 hours.
4. The method according to embodiment 1, wherein the stem cell derived cardiomyocytes are cultured with one or more anti-arrhythmic for between 24-48 hours.
5. The method according to embodiment 1, wherein the stem cell derived cardiomyocytes are cultured with one or more anti-arrhythmic for at least 48 hours.
6. The method according to embodiment 1, wherein the stem cell derived cardiomyocytes are cultured with one or more anti-arrhythmic for between 48-72 hours.
7. The method according to embodiment 1, wherein the stem cell derived cardiomyocytes are cultured with one or more anti-arrhythmic for at least 72 hours.
8. The method according to embodiment 1, wherein the stem cell derived cardiomyocytes are cultured with one or more anti-arrhythmic for between 72-96 hours.
9. The method according to embodiment 1, wherein the stem cell derived cardiomyocytes are cultured with one or more anti-arrhythmic for at least 96 hours.
10. The method according to any of the embodiments 1 to 9, wherein the antiarrhythmic agent is selected from the list of class I, class II, and class III antiarrhythmic agents or combinations thereof.
11. The method according to embodiment 10, wherein the class I antiarrhythmic agent is lidocaine or mexiletine, class II antiarrhythmic agent is metoprolol or propranolol, and/or class III antiarrhythmic agent is amiodarone or sotalol, or combination thereof.
12. The method according to embodiment 10, wherein the antiarrhythmic agent is selected from the list of class III in combination with class I and/or class II antiarrhythmic agents.
13. The method according to embodiment 11, wherein the antiarrhythmic agent is lidocaine and amiodarone.
14. The method according to embodiment 11, wherein the antiarrhythmic agent is mexiletine and sotalol.
15. The method according to embodiment 11, wherein the antiarrhythmic agent is metoprolol and sotalol.
16. The method according to embodiment 11, wherein the antiarrhythmic agent is amiodarone and propranolol.
17. The method according to embodiment 11, wherein the antiarrhythmic agent is lidocaine and sotalol.
18. The method according to embodiment 11, wherein the antiarrhythmic agent is amiodarone and metoprolol.
19. The method according to embodiment 11, wherein the antiarrhythmic agent is amiodarone and mexiletine.
20. The method according to embodiment 11, wherein the antiarrhythmic agent is sotalol and propranolol.
21. The method according to embodiment 10, wherein the antiarrhythmic agent is selected from the list of class II in combination with class I antiarrhythmic agents.
22. The method according to embodiment 11, wherein the antiarrhythmic agent is metoprolol and mexiletine.
23. The method according to embodiment 11, wherein the antiarrhythmic agent is lidocaine and metoprolol.
24. The method according to embodiment 11, wherein the antiarrhythmic agent is lidocaine and propranolol.
25. The method according to embodiment 11, wherein the antiarrhythmic agent is amiodarone and sotalol.
26. The method according to embodiment 11, wherein the antiarrhythmic agent is mexiletine and propranolol.
27. The method according to any one of the preceding embodiments, wherein the concentration of antiarrhythmic agent is at least at least 1 nM.
28. The method according to any of the preceeding embodiments, wherein the concentration of antiarrhythmic agent is in a range of 1 nM-100 nM.
29. The method according to embodiment 28, wherein the concentration of antiarrhythmic agent is about 1 nM.
30. The method according to embodiment 28, wherein the concentration of antiarrhythmic agent is about 10 nM.
31. The method according to embodiment 28, wherein the concentration of antiarrhythmic agent is about 100 nM.
32. The method according to any of the preceeding claims 1-26, wherein the concentration of antiarrhythmic agent is in a range of 0.1 μM-100 μM.
33. The method according to embodiment 32, wherein the concentration of antiarrhythmic agent is about 0.5 μM.
34. The method according to embodiment 32, wherein the concentration of antiarrhythmic agent is about 1 μM.
35. The method according to embodiment 32, wherein the concentration of antiarrhythmic agent is about 5 μM.
36. The method according to embodiment 32, wherein the concentration of antiarrhythmic agent is about 10 μM.
37. The method according to embodiment 32, wherein the concentration of antiarrhythmic agent is about 100 μM.
38. Antiarrhythmic cardiomyocyte cell population for use as a medicament.
39. Antiarrhythmic cardiomyocyte cell population for use in the treatment of heart failure.
40. Antiarrhythmic cardiomyocyte cell population for use in the prevention or alleviation of arrhythmia.
41. Antiarrhythmic cardiomyocyte cell population for use in the prevention or alleviation of proarrhythmia.
42. The antiarrhythmic cardiomyocyte cell population according to embodiments 38 to 41 for improvement in transplantation outcome of the transplanted stem cell-derived cardiomyocytes.
43. The antiarrhythmic cardiomyocyte cell population according to any one of embodiments 38 to 41 having reduction in coefficient of variation (CV) or beat to beat variability when compared to stem cell derived cardiomyocytes.
44. The antiarrhythmic cardiomyocyte cell population according to embodiment 43 having at least 50% reduction in coefficient of variation (CV) or beat to beat variability when compared to stem cell derived cardiomyocytes.
45. The antiarrhythmic cardiomyocyte cell population according to embodiment 43 having at least 70% reduction in coefficient of variation (CV) or beat to beat variability when compared to stem cell derived cardiomyocytes.
46. The antiarrhythmic cardiomyocyte cell population according to any one embodiments 38 to 45, having regulation of expression of gene selected from the list of GJA5, CACNA1G, NPPA and NPPB.
47. The antiarrhythmic cardiomyocyte cell population according to embodiment 46, having upregulation of GJA5 and/or CACNA1G.
48. The antiarrhythmic cardiomyocyte cell population according to embodiment to 46, having downregulation of NPPA and/or NPPB.
49. The antiarrhythmic cardiomyocyte cell population according to embodiment 46, having upregulation of GJA5 and/or CACNA1G and downregulation of NPPA and/or NPPB.
50. The antiarrhythmic cardiomyocyte cell population according to any one of embodiments 46 to 49, wherein the cell population has at least 1.5 times upregulation of GJA5, at least 2 times upregulation of CACNA1G, at least 2 times downregulation of NPPA and/or at least 4 times downregulation of NPPB when compared to stem cell-derived cardiomyocytes.
51. The antiarrhythmic cardiomyocyte cell population according to embodiment 50, wherein at least 10% of the cardiomyocytes have at least 1.5 times upregulation of GJA5, at least 2 times upregulation of CACNA1G, at least 2 times downregulation of NPPA and/or at least 4 times downregulation of NPPB when compared to stem cell-derived cardiomyocytes.
52. The antiarrhythmic cardiomyocyte cell population according to embodiment 50, wherein at least 20% of the cardiomyocytes have at least 1.5 times upregulation of GJA5, at least 2 times upregulation of CACNA1G, at least 2 times downregulation of NPPA and/or at least 4 times downregulation of NPPB when compared to stem cell-derived cardiomyocytes.
53. The antiarrhythmic cardiomyocyte cell population according to embodiment 50, wherein at least 40% of the cardiomyocytes have at least 1.5 times upregulation of GJA5, at least 2 times upregulation of CACNA1G, at least 2 times downregulation of NPPA and/or at least 4 times downregulation of NPPB when compared to stem cell-derived cardiomyocytes.
54. A kit comprising an antiarrhythmic agent and stem cell-derived cardiomyocytes.
55. The kit according to embodiment 54 for use in a method for the treatment of heart failure, preferably by the transplantation of the stem cell-derived cardiomyocytes.
56. The kit according to any one of embodiments 54 to 55, wherein the antiarrhythmic agent is selected from the list of class I, class II, class III, class IV, and class V antiarrhythmic agents, or a combination thereof.
57. The kit according to embodiment 56, wherein the antiarrhythmic agent is selected from the list of class I, class II, and class III antiarrhythmic agents, or a combination thereof.
58. The kit according to embodiment 56, wherein the class I antiarrhythmic agent is lidocaine or mexiletine, the class II antiarrhythmic agent is metoprolol or propranolol, and/or the class III antiarrhythmic agent is amiodarone or sotalol, or a combination thereof.
59. The kit according to embodiment 58, comprising amiodarone and lidocaine.
60. The kit according to embodiment 58, comprising mexiletine and sotalol.
61. The kit according to embodiment 58, comprising metoprolol and sotalol.
62. The kit according to embodiment 58, comprising metoprolol and mexiletine.
63. The kit according to embodiment 58, comprising amiodarone and propranolol.
64. A composition comprising stem cell derived cardiomyocytes, one or more antiarrhythmic agents and optionally a biomaterial for use in the treatment of heart failure.
65. Antiarrhythmic agent for use in a method for the treatment of heart failure by transplantation of stem cell-derived cardiomyocytes.
66. Antiarrhythmic agent according to embodiment 65, wherein the antiarrhythmic agent is selected from the list of antiarrhythmic agents of class I, class II, class III, class IV, and class V.
67. Antiarrhythmic agent according to embodiment 65, wherein the antiarrhythmic agent is selected from the list of antiarrhythmic agents of class I, class II, and class III.
68. Antiarrhythmic agent according to any one of embodiments 65 to 67, wherein the antiarrhythmic agent is a class III antiarrhythmic agent.
69. Antiarrhythmic agent according to embodiment 68, wherein the class III antiarrhythmic agent is amiodarone.
70. Antiarrhythmic agent according to embodiment 68, wherein the class III antiarrhythmic agent is sotalol.
71. Amiodarone for use in a method for the treatment of heart failure by transplantation of stem cell-derived cardiomyocytes.
72. Sotalol for use in a method for the treatment of heart failure by transplantation of stem cell-derived cardiomyocytes.
73. Antiarrhythmic agent according to any one of embodiments 65 to 67, wherein the antiarrhythmic agent is a class II antiarrhythmic agent.
74. Antiarrhythmic agent according to embodiment 73, wherein the class antiarrhythmic agent is metoprolol.
75. Antiarrhythmic agent according to embodiment 73, wherein the class antiarrhythmic agent is propranolol 76. Metoprolol for use in a method for the treatment of heart failure by transplantation of stem cell-derived cardiomyocytes.
77. Propranolol for use in a method for the treatment of heart failure by transplantation of stem cell-derived cardiomyocytes.
78. Antiarrhythmic agent according to any one of embodiments 65 to 67, wherein the antiarrhythmic agent is a class I antiarrhythmic agent.
79. Antiarrhythmic agent according to embodiment 78, wherein the class I antiarrhythmic agent is lidocaine.
80. Antiarrhythmic agent according to embodiment 78, wherein the class I antiarrhythmic agent is mexiletine.
81. Lidocaine for use in a method for the treatment of heart failure by transplantation of stem cell-derived cardiomyocytes.
82. Mexiletine for use in a method for the treatment of heart failure by transplantation of stem cell-derived cardiomyocytes.
83. Antiarrhythmic agent according to any one of embodiments 65 to 67, wherein the antiarrhythmic agent is a combination comprising a class III and a class I antiarrhythmic agent.
84. Antiarrhythmic agent according to embodiment 83, wherein the antiarrhythmic agent is a combination comprising amiodarone and a class I antiarrhythmic agent.
85. Antiarrhythmic agent according to embodiment 83, wherein the antiarrhythmic agent comprises amiodarone and lidocaine.
86. Antiarrhythmic agent according to embodiment 83, wherein the antiarrhythmic agent comprises amiodarone and mexiletine.
87. Antiarrhythmic agent according to embodiment 83, wherein the antiarrhythmic agent is a combination comprising sotalol and a class I antiarrhythmic agent.
88. Antiarrhythmic agent according to embodiment 83, wherein the antiarrhythmic agent comprises sotalol and mexiletine.
89. Antiarrhythmic agent according to embodiment 83, wherein the antiarrhythmic agent comprises sotalol and lidocaine.
90. Antiarrhythmic agent according to any one of the embodiments 65 to 89 for obtaining a high probability of successful transplantation outcome of the transplanted stem cell-derived cardiomyocytes.
91. Antiarrhythmic agent according to any one of the embodiments 65 to 89 for facilitating integration of the transplanted stem cell-derived cardiomyocytes into a host myocardium.
92. Antiarrhythmic agent according to any one of embodiments 65 to 89 for improving beating and/or rhythm of the transplanted stem cell-derived cardiomyocytes.
93. Antiarrhythmic agent according to any one of embodiments 65 to 89, wherein the antiarrhythmic agent regulates the expression of a gene selected from the list of GJA5, CACNA1G, NPPA, NPPB.
94. Antiarrhythmic agent for use in a method for the treatment or prevention of arrhythmia caused by the transplantation of stem cell-derived cardiomyocytes.
95. Antiarrhythmic agent according to embodiment 94, wherein the arrhythmia is non-sustained ventricular tachycardia, sustained ventricular tachycardia and sustained accelerated idioventricular rhythm.
96. Antiarrhythmic agent for use in a method for the prevention of graft rejection following the transplantation of stem cell-derived cardiomyocytes.
97. Antiarrhythmic agent according to embodiment 94, wherein the arrhythmia is caused by a method for the treatment of heart failure by transplantation of stem cell-derived cardiomyocytes.
98. Composition comprising a class I antiarrhythmic agent and a class III antiarrhythmic agent for use in a method for the treatment of heart failure by transplantation of stem cell-derived cardiomyocytes.
99. Composition according to embodiment 98, wherein the class I antiarrhythmic agent is amiodarone, and the class III antiarrhythmic agent is lidocaine.
100. Use of an antiarrhythmic agent in a method for the treatment of heart failure by transplantation of stem cell-derived cardiomyocytes.
101. Antiarrhythmic agent and stem cell-derived cardiomyocytes for use in a method for the treatment of heart failure by transplantation of the stem cell-derived cardiomyocytes.
102. Antiarrhythmic agent and stem cell-derived cardiomyocytes for use in a method for the prevention of arrhythmia in the treatment of heart failure by transplantation of the stem cell-derived cardiomyocytes.
103. A method of regulating gene expression in stem cell-derived cardiomyocytes for obtaining a high probability of successful transplantation outcome, comprising contacting the stem cell-derived cardiomyocytes with an antiarrhythmic agent.
104. The method according to embodiment 103, wherein the antiarrhythmic agent is selected from the list of class I, class II, class III, class IV, and class V antiarrhythmic agents, or a combination thereof.
105. The method according to embodiment 104, wherein the antiarrhythmic agent is selected from the list of class I, class II, and class III antiarrhythmic agents, or a combination thereof.
106. The method according to embodiment 105, wherein the class I antiarrhythmic agent is lidocaine, the class II antiarrhythmic agent is metoprolol, and/or the class III antiarrhythmic agent is amiodarone, or a combination thereof.
107. A method for obtaining stem cell-derived cardiomyocytes with a high probability of successful transplantation outcome, comprising a step of regulating the expression of a gene selected from the list of GJA5, CACNA1G, NPPA and NPPB.
108. The method according to embodiment 107, wherein the gene GJA5 is upregulated at least 1.5 times and/or the gene CACNA1G is upregulated at least 2 times and/or the gene NPPA is downregulated at least 2 times and/or the gene NPPB is downregulated at least 4 times.
109. The method according to any one of embodiments 107 to 108, wherein the step of regulating the expression of the gene is carried out by contacting the stem cell-derived cardiomyocytes with an antiarrhythmic drug.
110. The method according to any one of embodiments 107 to 108, wherein the regulation of the gene expression is in vitro.
111. Antiarrhythmic agent, composition, use, kit or method according to any one of the preceding embodiments, wherein the stem cell-derived cardiomyocytes are derived from human pluripotent stem cells, such as human embryonic stem cells.
112. A method for the treatment of heart failure, comprising the steps of:
   a) obtaining in vitro stem cell-derived cardiomyocytes,
   b) transplanting the stem cell-derived cardiomyocytes into a patient, and
   c) co-administering an antiarrhythmic agent to the patient.
113. The method according to embodiment 112, further comprising the step of:
   contacting in vitro the stem cell-derived cardiomyocytes obtained in step a) with an antiarrhythmic agent to obtain an antiarrythmic cardiomyocyte cell population and transplanting said antiarrythmic cardiomyocyte cell population to the patient.
114. The method according to any one of embodiments 112 and 113, wherein the antiarrhythmic agent comprises amiodarone and lidocaine.
115. The method according to any one of embodiments 112 and 113, wherein the antiarrhythmic agent comprises mexiletine and sotalol.
116. The method according to any one of embodiments 112 and 113, wherein the antiarrhythmic agent comprises metoprolol and sotalol.
117. The method according to any one of embodiments 112 and 113, wherein the antiarrhythmic agent comprises metoprolol and mexiletine.
118. The method according to any one of embodiments 112 and 113, wherein the antiarrhythmic agent comprises amiodarone and propranolol.
119. Antiarrhythmic agent for use in a method according to any one of embodiments 112 to 118.

### EXAMPLES

### Example 1

In order to determine the effect of anti-arrythmic agents on stem-cell derived cardiomyocytes beyond the well-established immediate changes in electrophysiological response by modulation of ion-channel activity, we analysed changes in gene expression resulting from long-term exposure (>24 h) of the cardiomycotyes to the anti-arryhtmic agents. For this purpose, we evaluated the impact of commonly used anti-arrhythmic agents like amiodarone, licodaine, on gene associated with electrical signal propagation, modulation of cardiac hypertrophy, calcium handling and cardiomyocyte maturation.

Experimental Procedure Human embryonic stem cells (hESCs) were maintained under feeder-free conditions on LN521 (BioLamina) in iPSBrew (Miltenyi). The cells were passaged every 3-4 days using accutase (Innovative Cell Technology) and seeded in iPSBrew supplemented with 10 μM Y-27632 (Sigma) at 1.6-2.4×10⁴ cells/cm². Cell lines were tested negative for mycoplasma contaminations and karyotypic abnormalities throughout this study.

Cells were differentiated towards cardiomyocytes in an adapted 3D suspension protocol (Kempf H et al Bulk cell density and Wnt/TGFbeta signalling regulate mesendodermal patterning of human pluripotent stem cells. Nat Commun. 2016; 7:13602). In brief, cells were inoculated in 6-well suspension plates (Greiner) for aggregate formation at 0.16×10⁶ cells/mL in iPSBrew supplemented with 10 μM Y-27632. After 2 days, differentiation was induced using 4-8 μM CHIR99021 (Tocris) for 24 h followed by 2 μM Wnt-C59 (Tocris) for 48 h in RPMI1640 medium (Life Technologies) supplemented with 2% B27 without insulin (Life Technologies) or RPMI1640 medium supplemented with 0.5 mg/mL human recombinant albumin (ScienceCell) and 0.2 mg/mL L-ascorbic acid 2-phosphate (Sigma). Cells were kept in RPMI1640 supplemented with 2% B27 from day 5 onwards.

Obtained cardiomyocytes were dissociated into single cells after 10-15 days of differentiation using STEMdiff Cardiomyocyte dissociation kit (Stem Cell Technologies) according to manufacturer's instruction for further characterization, functional analysis and transplantation experiments.

### Evaluation of Antiarrhythmic Drugs

Dissociated cardiomyocytes were seeded in RPMI1640 medium supplemented with 2% B27 and 0.1% Pen/Strep (Gibco) on laminin-521 or geltrex (Life Technologies)-coated plates at a cell density of 1×10⁵/cm². After 4 days, cardiomyocytes were exposed to antiarrhythmic drugs for at least 72 h at the following concentrations: 1 μM, 10 μM and 100 μM amiodarone, 0.1 μM, 1 μM and 10 μM metoprolol and 0.1 μM, 1 μM and 10 μM lidocaine (all Sigma) and combinations thereof at each concentration. Blank medium, addition of solvents as well as cardiomyocytes cultured for 9 days and day 42 were used as control. Beating of cells was assessed after 48 h, 72 h and 96 h. Cells were harvested following incubation for 10 minutes in RLTplus buffer (Qiagen). Changes in gene expression were determined using a custom nanostring gene panel (NanoString Technologies) according to manufacturer's instruction for the following target sequences including 7 housekeeping genes listed in Table 1.

TABLE 1

| | |
|---|---|
| ACTA2 | ATTCCTTCGTTACTACTGCTGAGCGTGAGATTGTC |
| (NM_001613.1): | CGGGACATCAAGGAGAAACTGTGTTATGTAGCTCT |
| | GGGACTTTGAAAATGAGATGGCCACTGCCGC |
| CACNA1G | TTTGACAACATTGGCTATGCCTGGATCGCCATCTT |
| (NM_198397.1): | CCAGGTCATCACGCTGGAGGGCTGGGTCGACATCA |
| | TGTACTTTGTGATGGATGCTCATTCCTTCT |
| GJA5 | AACATCTGTCACCCTGCAGCTCCTTTACAGTTCAA |
| (NM_005266.5): | TCCAATGATAGAAACCATCCCTTCCCTTTCTCCCT |
| | TGGCTGTTCACCCAGCCATTCCCTGAAGGC |
| NKX2-5 | GCGCTGCCACCATGTTCCCCAGCCCTGCTCTCACG |
| (NM_004387.3): | CCCACGCCCTTCTCAGTCAAAGACATCCTAAACCT |
| | GGAACAGCAGCAGCGCAGCCTGGCTGCCGC |
| NPPA | ACCGTGAGCTTCCTCCTTTTACTGGCATTCCAGCT |
| (NM_006172.2): | CCTAGGTCAGACCAGAGCTAATCCCATGTACAATG |
| | CCGTGTCCAACGCAGACCTGATGGATTTCA |
| NPPB | GGCGGCATTAAGAGGAAGTCCTGGCTGCAGACACC |
| (NM_002521.2): | TGCTTCTGATTCCACAAGGGGCTTTTTCCTCAACC |
| | CTGTGGCCGCCTTTGAAGTGACTCATTTTT |
| SCN5A | TGGCTGTCACCTTTTTAATTTCCAGAACTGCACAA |
| (NM_198056.2): | TGACCAGCAGGAGGGAAGGACAGACATCAAGTGCC |
| | AGATGTTGTCTGAACTAATCGAGCACTTCT |
| TNNT2 | CAACGATAACCAGAAAGTCTCCAAGACCCGCGGGA |
| (NM_ | AGGCTAAAGTCACCGGGCGCTGGAAATAGAGCCTG |
| 001276346.1): | GCCTCCTTCACCAAAGATCTGCTCCTCGCT |
| ACTB | TGCAGAAGGAGATCACTGCCCTGGCACCCAGCACA |
| (NM_001101.2): | ATGAAGATCAAGATCATTGCTCCTCCTGAGCGCAA |
| | GTACTCCGTGTGGATCGGCGGCTCCATCCT |
| EMC7 | TGCTGAATTCCAACCATGAGTTGCCTGATGTTTCT |
| (NM_020154.2): | GAGTTCATGACAAGACTCTTCTCTTCAAAATCATC |
| | TGGCAAATCTAGCAGCGGCAGCAGTAAAAC |
| GUSB | CCGATTTCATGACTGAACAGTCACCGACGAGAGTG |
| (NM_000181.3): | CTGGGGAATAAAAAGGGGATCTTCACTCGGCAGAG |
| | ACAACCAAAAAGTGCAGCGTTCCTTTTGCG |
| HSP90AB1 | AGCCAATATGGAGCGGATCATGAAAGCCCAGGCAC |
| (NM_007355.2): | TTCGGGACAACTCCACCATGGGCTATATGATGGCC |
| | AAAAAGCACCTGGAGATCAACCCTGACCAC |
| PPA1 | ATACTGGCTGTTGTGGTGACAATGACCCAATTGAT |
| (NM_021129.3): | GTGTGTGAAATTGGAAGCAAGGTATGTGCAAGAGG |
| | TGAAATAATTGGCGTGAAAGTTCTAGGCAT |
| TBP | ACAGTGAATCTTGGTTGTAAACTTGACCTAAAGAC |
| (NM_ | CATTGCACTTCGTGCCCGAAACGCCGAATATAATC |
| 001172085.1): | CCAAGCGGTTTGCTGCGGTAATCATGAGGA |
| TFRC | CAGTTTCCACCATCTCGGTCATCAGGATTGCCTAA |
| (NM_003234.1): | TATACCTGTCCAGACAATCTCCAGAGCTGCTGCAG |
| | AAAAGCTGTTTGGGAATATGGAAGGAGACT |

### Results

To study the direct impact of antiarrhythmic drugs on the properties of hESC-derived cardiomyocytes, changes in expression level of selected genes were analyzed that are associated with action potential formation (HCN1, HCN4, KCNA5, KCNE4, KCNH7, KCNJ3, KCNJ5, SCN1B, SCN5A), electrical signal propagation (GJA1, GJA5, GJD3), calcium handling (CACNA1C, CACNA1D, CACNA1G, RYR2, PLN), cardiac maturation (HOPX, MYH7, MYL2, TNN13) and cardiac hypertrophy (NPPA, NPPB) as well as pan-cardiomyocyte markers (NKX2-5, TNNT2, ACTA2).

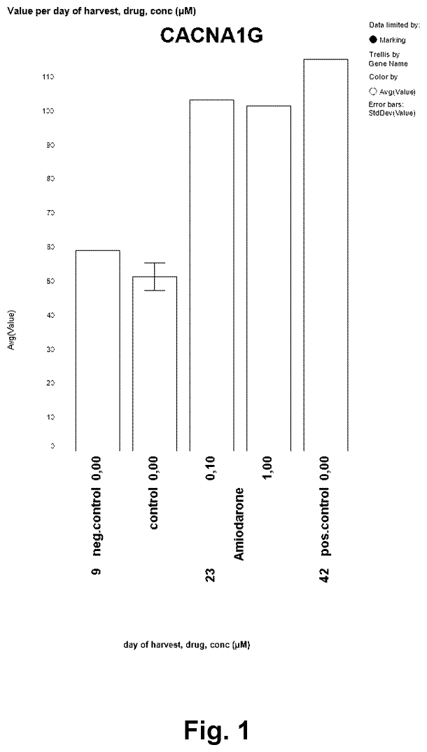

Strikingly, 5-day treatment of hES-derived cardiomyocytes with 0.1 μM and 1 μM amiodarone induced a 2-fold increase in expression of the T-type voltage-dependent calcium channel a-subunit 1G CACNA1G compared to untreated controls on day 23 and early-stage (immature) cardiomyocytes on day 9 (FIG. 1). T-type Ca²⁺ channels are expressed in the developing fetal ventricular myocytes (Cribbs L L et al, Identification of the t-type calcium channel (Ca(v)3.1d) in developing mouse heart. Circ Res. 2001; 88(4):403-7) and play a key role in modulating the intracellular distribution of the second messenger Ca²⁺ by regulation of Ca²⁺ entry from internal stores. Thereby, the channel modulates a variety of cellular processes, including the beating of cardiomyocytes. More specifically CACNA1G controls electrical and pacing activity in the heart. Importantly, malfunctioning of the cannel is associated with arrhythmias both the atria as well as ventricle, particularly in the failing heart (Perez-Reyes E. Molecular physiology of low-voltage-activated t-type calcium channels. Physiol Rev. 2003; 83(1):117-61) (Vassort G, Talavera K, Alvarez J L. Role of T-type Ca2+ channels in the heart. Cell Calcium. 2006; 40(2):205-20). The clear upregulation of CACNA1G by amiodarone thus suggests an increased capability of the treated hESC-derived cardiomyocytes in their inherent electrophysiological capacitance to control and prevent arrhythmic responses.

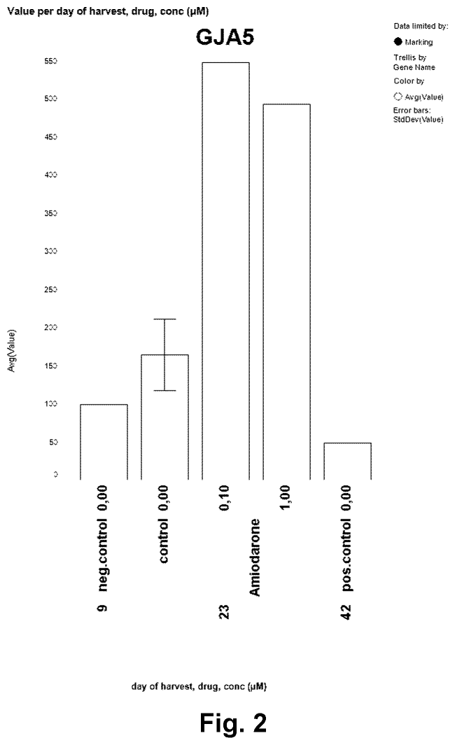

Likewise, 0.1 μM and 1 μM amiodarone resulted in a >3-fold upregulation of the gene encoding for the high-conductance gap junction protein GJA5 (FIG. 2). GJA5 is expressed in the early ventricle as well as the ventricular conduction system (Delorme B et al, Developmental regulation of connexin 40 gene expression in mouse heart correlates with the differentiation of the conduction system. Dev Dyn. 1995; 204(4):358-71) and represents a key player in the conduction of the electrical current across the ventricles (Shekhar A et al, Transcription factor ETV1 is essential for rapid conduction in the heart. J Clin Invest. 2016; 126(12):4444-59). Several somatic mutations in GJA5 are associated with proarrhythmic properties of the myocardium, including ventricular arrhythmias (Delmar M, Makita N. Cardiac connexins, mutations and arrhythmias. Curr Opin Cardiol. 2012; 27(3):236-41). Consequently, upregulation of GJA5 in ES-derived cardiomyocytes by amiodarone is likely to accelerate electrical signal propagation across cell-cell contacts and thereby suppressing arrhythmic behavior, particularly via macro or micro-reentries, thereby reducing the risk of the occurrence of ectopic beating foci.

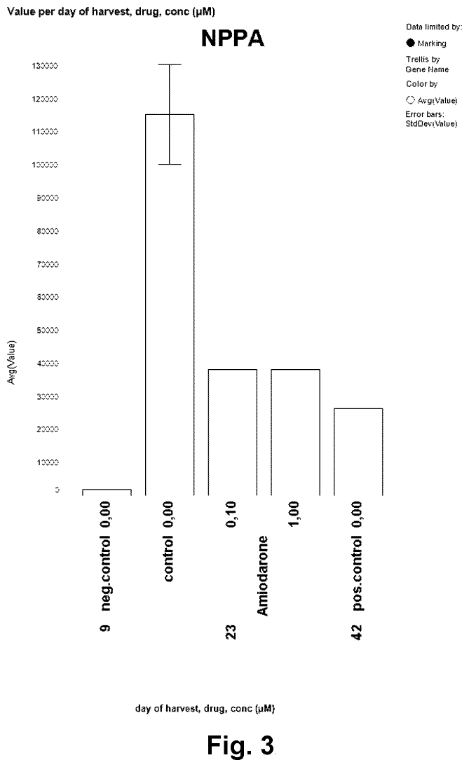

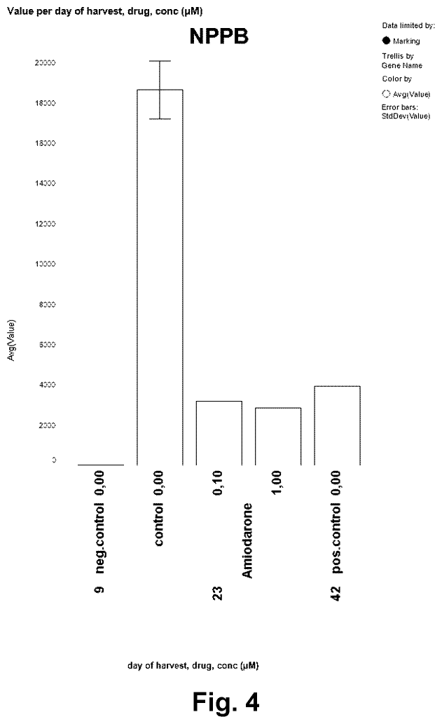

In contrast to the increased levels of CACNA1G and GJA5, treatment with amiodarone resulted in about 3-fold and 5-fold reduction in NPPA and NPPB expression, respectively (FIGS. 3 and 4). NPPA and NPPB encode the secreted hormone ANP (atrial natriuretic peptide) and BNP (Brain natriuretic peptide), primarily secreted from the atria and less prominently the ventricles of the adult heart in the response to mechanical stretch. Quantification of natriuretic peptide levels are routinely used as a tool for the diagnosis of heart failure (McMurray J J et al Guidelines ESCCfP. ESC Guidelines for the diagnosis and treatment of acute and chronic heart failure 2012: The Task Force for the Diagnosis and Treatment of Acute and Chronic Heart Failure 2012 of the European Society of Cardiology. Developed in collaboration with the Heart Failure Association (HFA) of the ESC. Eur Heart J. 2012; 33(14):1787-847). Interestingly, both ANP and BNP are involved in modulating cardiac electrophysiology (Perrin M J, Gollob M H. The role of atrial natriuretic peptide in modulating cardiac electrophysiology. Heart Rhythm. 2012; 9(4):610-5). In particular, elevated BNP levels are associated with increased arrhythmic events in patients with left ventricular dysfunction (Galante O et al, Brain natriuretic peptide (BNP) level predicts long term ventricular arrhythmias in patients with moderate to severe left ventricular dysfunction. Harefuah. 2012; 151(1):20-3, 63, 2). However, the exact mechanisms on how natriuretic peptides regulate electrophysiological action in humans remains uncertain. It is believed that the peptides induce action potential shortening and thereby increasing the likelihood of reentries. Furthermore, a prolonged action potential duration by reduced ANP and/or BNP levels will decrease the likelihood of tachyarrhythmias. Thus, lowering NPPA and NPPA using amiodarone in hESC-derived cardiomyocytes reduces the risk of graft-induced arrhythmias as well as tachycardias and occurrence of ectopic beating foci from hESC-derived cardiomyocytes following cardiac transplantation.

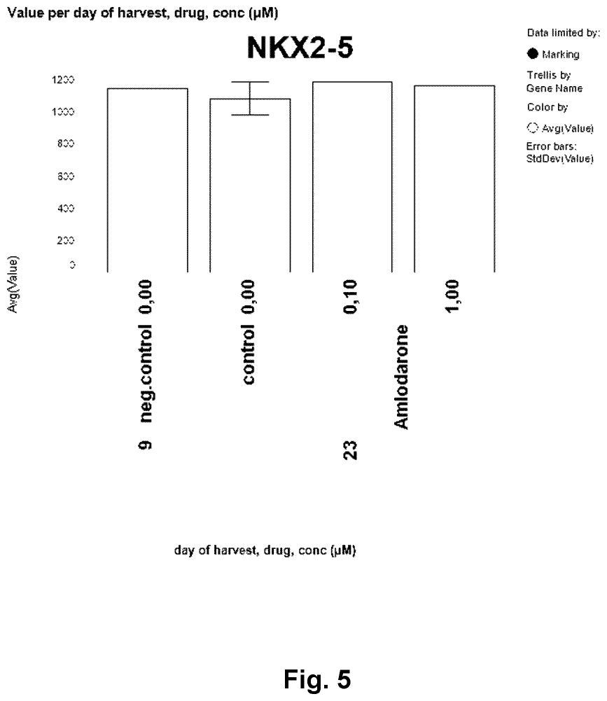

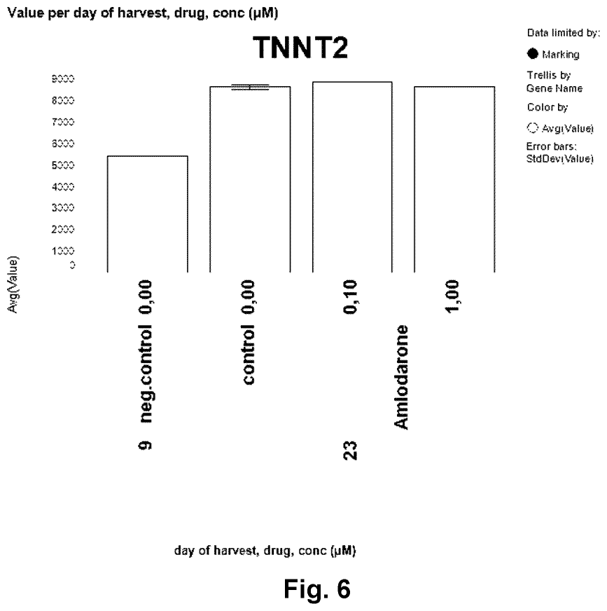

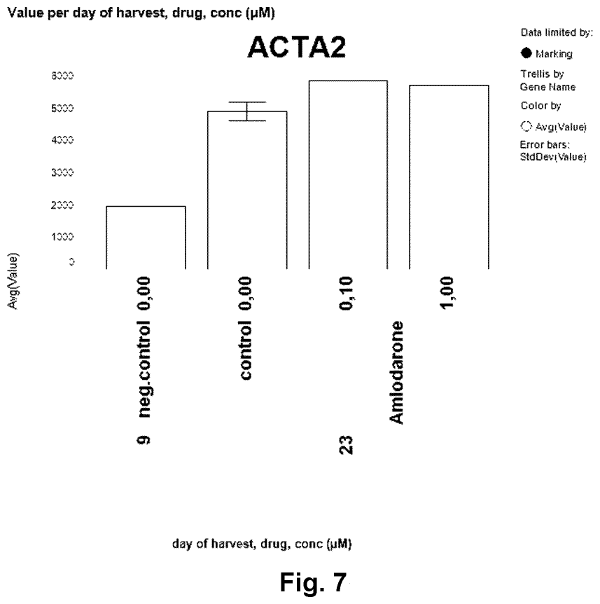

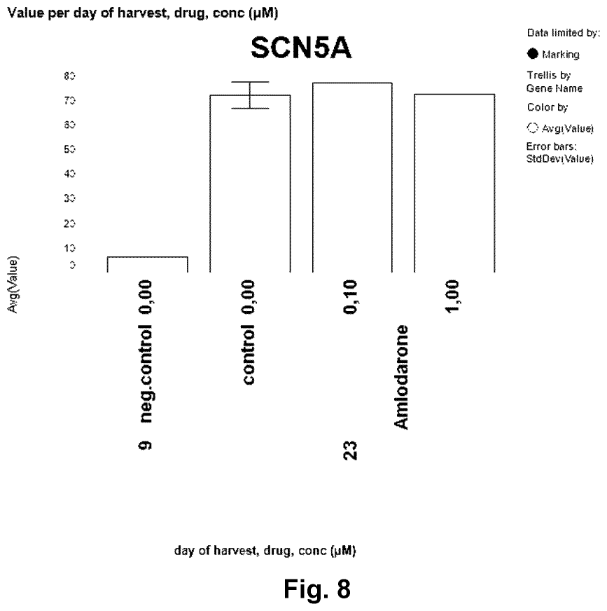

It is noteworthy, that the beating frequency of hESC-derived cardiomyocytes was clearly reduced or abrogated at 1 μM and 10 μM, respectively, reducing the probability of tachyarrhythmias to occur. At the same time, amiodarone did not result in changes of the expression of pan-cardiomyocyte genes, including NKX2-5, TNNT2 and ACTA2 (Figure. 5-7), suggesting that the overall cardiomyocyte identity is not affected. Also, it had no effect on the expression of other ion channels including SCN5A (FIG. 8), one of its targets (Honjo H et al, Block of cardiac sodium channels by amiodarone studied by using Vmax of action potential in single ventricular myocytes. Br J Pharmacol. 1991; 102(3):651-6) that regulate the upstroke velocity of the action potential in the human heart.

Overall, exposing hESC-derived cardiomyocytes to amiodarone induces a unique expression profile comprising elevated levels of CACNA1G and GJA5 accompanied by decreased NPPA and NPPB. This imparts amiodarone-treated cardiomyocytes distinct electrophysiological features, including increased capacities to control intracellular calcium levels, faster signal conduction across the cardiac tissue, and decreased sensitivity to arrhythmic events and tachycardias. Consequently, these (modified) anti-arrhythmic cardiomyocyte cell population provides a superior cell source to regenerate the heart by omitting graft-induced arrhythmias and/or tachycardias.

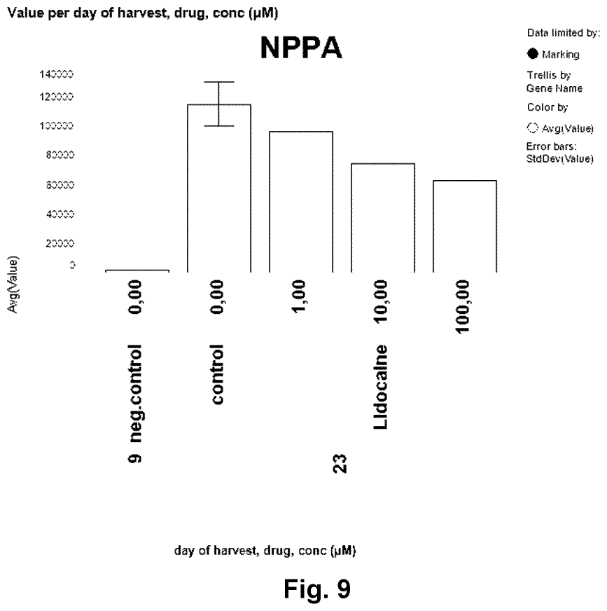

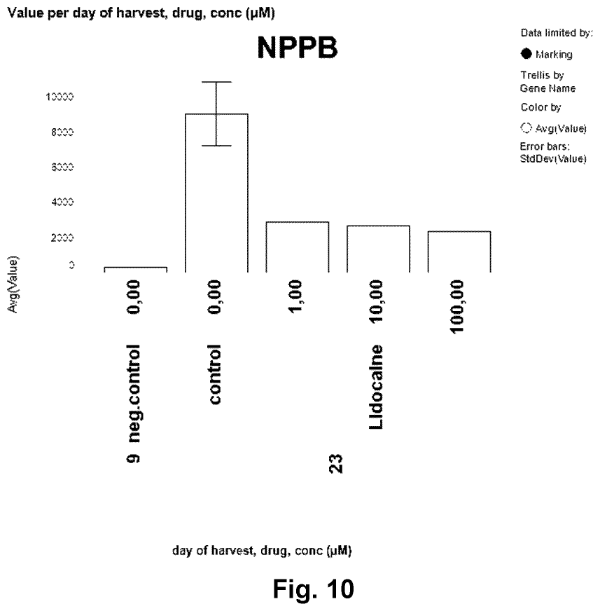

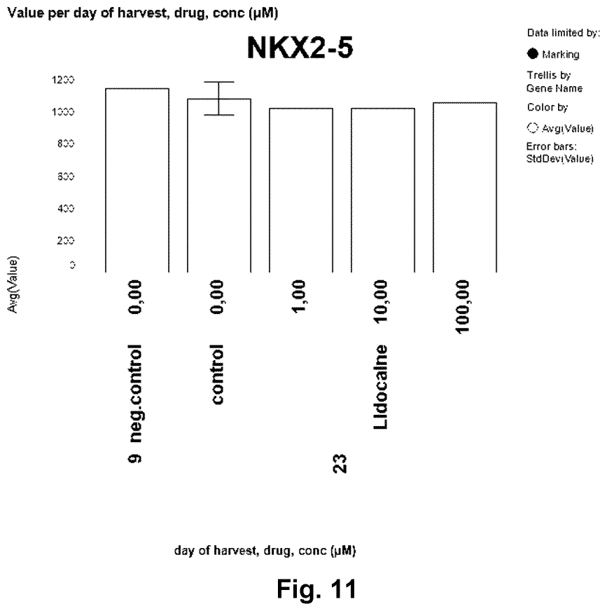

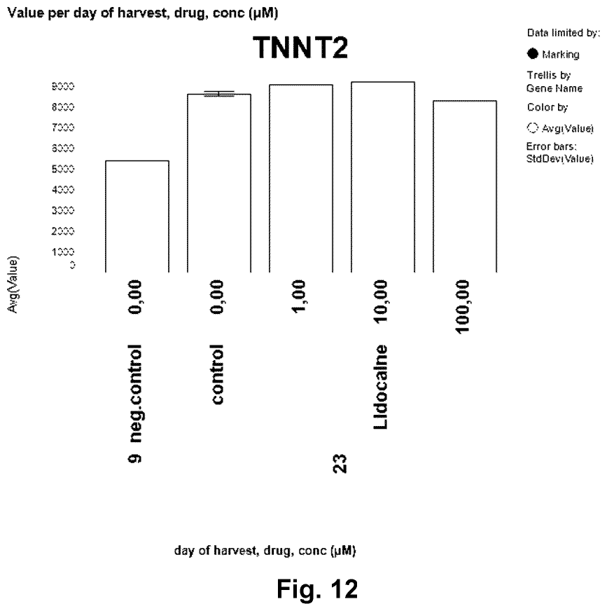

Similarly, other classes of anti-arrhythmic drugs were found to modulate NPPA and NPPB. Lidocaine, the most relevant class-1b antiarrhythmic drug decreased both natriuretic peptides at 1 μM, 10 μM and 100 μM in a concentration dependent manner (FIGS. 9, 10), without effecting the expression of pan-cardiomyocyte markers (NKX2-5 and TNNT2; FIGS. 11, 12). Considering the overall relevance of NPPA and NPPB on regulating the electrophysiological behavior of cardiomyocytes, treatment of hESC-derived cardiomyocytes using lidocaine represents an additional promising strategy to avoid graft-induced side effects in cardiac transplantations.

Together, our results show an unexpected impact of anti-arrhythmic agents on the gene expression of stem-cell derived cardiomyocytes, resulting in a anti-arrhythmic cardiomyocyte cell population having modified expression pattern of genes associated with cardiac hypertrophy, calcium handling and electrical signal conduction, all relevant classes of genes that are relevant in regulating the electrophysiological behaviour of cardiomycytes.

### Example 2

In order to verify the lasting effect and the robustness across different culture systems, we tested the effect of the antiarrhythmic agents on the gene expression profile of the stem-cell derived cardiomyocytes using 3D suspension aggregates and measured the gene expression 48 h after removal of the anti-arrhythmic agents.

### Experimental Procedure

The experiment was conducted as described in Example 1 with the following modifications. Instead of dissociating and seeding the stem-cell derived cardiomyocytes in a two-dimensional monolayer, the cells were maintained as three-dimensional suspension aggregates, directly obtained 14 days after induction of the cardiac differentiation. The aggregates were subsequently maintained in 6-well suspension plates on an orbital shaker (75 rpm) at a cell density of about 1.5×10⁶ cells/ml in 3 mL medium. Cells in aggregates were exposed to 10 μM amiodarone for about 120 h. Thereafter the cells were maintained for additional 48 h in RPMI medium supplemented with 2% B27+0.1% P/S. A full medium exchange was conducted every 48-72 hours. Cells were subsequently harvested and subjected to RNA expression analysis.

### Results

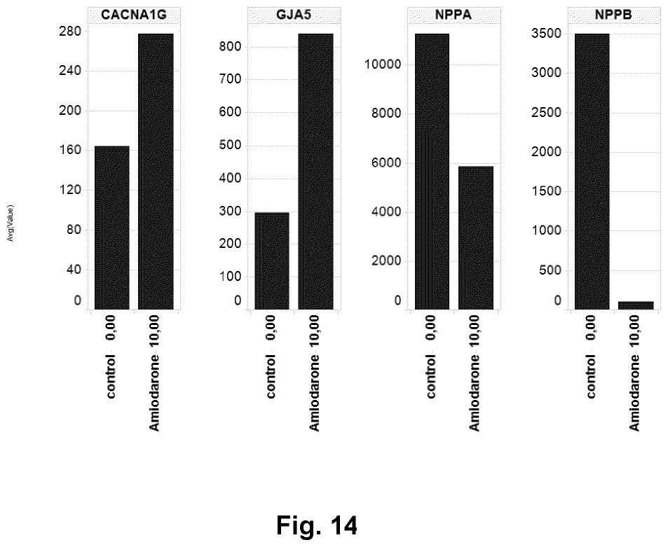

The gene expression profile of the aggregates measured 48 h after treatment with 10 μM amiodarone show an about 1.75 fold increase in CACNA1G, about 2.7-fold increase in GJA5 and an about 2-fold and more than 15-fold decrease in NPPA and NPPB, respectively (FIG. 14).

The results thus confirm sustained and clear effects of anti-arrhythmic agents, e.g. amiodarone on gene expression level that are associated with the modified electrophysiological properties of the stem-cell derived cardiomyocytes that result in reduced arrhythmic potential as shown in Example 3 and 4. In addition the results show that the effect is independent of the culture format, e.g. is induced in two-dimensional monolayer cultures as well as three-dimensional suspension cultures that more closely resemble in vivo tissues. The effect of the anti-arrhythmic agents is thus expected to translate in in vivo applications.

### Example 3

In order to determine the lasting impact on the electrophysiological properties of the stem-cell derived cardiomyocytes following exposure to anti-arrhythmic agents, we conducted functional cardiomyocyte testing by means of Ca²⁺-recordings and determined the arrhythmogenic potential of the stem-cell derived cardiomyocyte population after exposure to the anti-arrhythmic agents. Beat-to-beat variability was used as an in vitro surrogate readout for the in vivo (pro)arrhythmic potential of stem-cell derived cardiomyocytes (Rosanne Varkevisser et al, Beat-to-beat variability of repolarization as a new biomarker for proarrhythmia in vivo, Heart Rhythm Volume 9, Issue 10, October 2012, Pages 1718-1726); (Kazuto Yamazaki et al, Beat-to-Beat Variability in Field Potential Duration in Human Embryonic Stem Cell-Derived Cardiomyocyte Clusters for Assessment of_Arrhythmogenic Risk, and a Case Study of Its Application, Pharmacology & Pharmacy, Vol. 5 No. 1, 2014, pp. 117-128).

Of note, all Ca²⁺-recordings were conducted at least 24 h after exposure to exclude the known direct effects of the agents via ion channel modulation.

### Experimental Procedure

Ca²⁺-recordings were performed on human induced pluripotent stem cell derived cardiomyocytes (stem cell-derived cardiomyocytes) from Fujifilm Cellular Dynamics, USA (FCDI; iCell2 cardiomyocytes, Donor No. 01434, Lot No 105170).

The cells were delivered as frozen vials and stored until use in liquid nitrogen. All culturing media necessary for thawing and culturing was supplied by the cell supplier. Thawing, plating and cultivation procedures were followed according to the protocol supplied by the manufacturer. Cells were plated directly onto the 384-well Greiner μClear plates coated with fibronectin. Plating density was 17.500 cell/well in a final volume of 50 μl. Media was changed (90%) one day after plating and then every second day and 3 hours before the experiments for the pilot study. For the compound experiments, the compounds were added at day-in-vitro (DIV) 2, followed by a compound-containing media exchange (90%) at DIV4. Treatment was ended by exchanging medium to standard cultivation medium on DIV 6. Recording took place at DIV 7. Experiments were performed at a minimum of n=10 for each compound concentration.

For Ca²⁺ imaging experiments media was exchanged for the recordings by a HEPES buffered recording solution. A fluorescent Ca²⁺ indicator (Cal-520-AM) was applied at a concentration of 2 μM and allowed to accumulate in the cells for 30 min before the buffer was exchanged again to dye-free buffer. Cells were allowed to recover for 10 min at 37° C. in the Hamamatsu FDSS recording system. All experiments were performed at 37° C. The framerate of the camera was set to a minimum of 35 Hz for recording and a binning of 4×4. To assess if the quality was sufficient for the experiments several parameters were reviewed (by-eye inspection), including regularity of the beating, shape and amplitude of the Ca²⁺ signals and variability of these parameters between the different wells. Since the cells were spontaneously active, no electrical stimulation was applied.

Cells were recorded for 5 min prior to compound application. 200 μM Moxifloxacin was applied after the baseline phase in a single-concentration-per-well fashion, followed by a wash-in phase of 5 min. Fluorescent activity was then recorded for additional 5 min. Moxifloxacin without compound preincubation was included as control (n=18), distributed over multiple plates.

The beat-to-beat variability measured as coefficient of variation (CV) of the time interval between consecutive transient Ca²⁺ transients within the respective recording epoch was analyzed, using FDSSv3.4 Offline, followed by further analysis and compilation of the plots using Igor Pro 8.0.4.2 (Wavemetrics, USA).

Coefficient of variation was calculated as

$$C V = \frac{σ}{μ}$$

where σ denotes the standard deviation and p represents the mean.

The SEM was calculated as ratio between standard deviation and the square root of the number of experiments:

$$S E M = \frac{σ}{\sqrt{n}}$$

where σ denotes the standard deviation and n is the number of experiments.

### Results

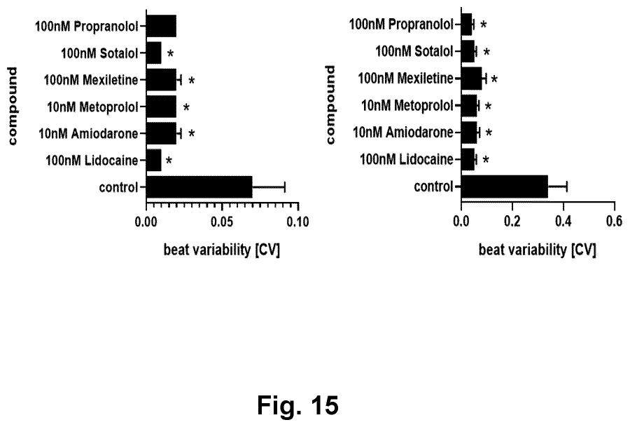

The coefficient of variation (CV) indicating the beat-to-beat variability of the antiarrythmic cardiomyocyte cell population at the indicated concentrations were compared to control treatment. The results show a clear reduction of beat-to-beat variability reflected by the coefficient of variation (CV) under baseline conditions as well as after induction of proarrhythmic conditions using moxifloxacine for all tested compounds comprising 3 different classes of anti-arrhythmic drugs namely class I (e.g. Lidocaine or Mexiletine), class II (e.g. Propranolol, Metoprolol) and class III (e.g. Amiodarone, Sotalol) (FIG. 15). The CV was reduced after exposure to 100 nM Lidocaine by 80.8% under baseline conditions and 84.7% under proarrhythmic conditions compared to the respective control conditions that were not exposed to an anti-arrhythmic agent. Similarly, 10 nM amiodarone reduced CV by 76.5% and 83.3%, 10 nM metoprolol by 79.2% and 83.2%, 100 nM Mexiletine by 76.79% and 77.5%, 100 nM Sotalol by 79.4% and 84.9%, and 100 nM propranolol by 73.7% and 88.5%, under baseline and anti-arrhythmic conditions, respectively. Notably, this clear reduction in beat-to-beat variability was observed 24 h after withdrawal of the compounds and is thus not dependent on the continuous presence of the drug.

Overall, the results clearly show the reduction in beat-to-beat variability following the treatment with the class I, or II anti-arrhythmic agents with an overall reduction in CV of 70-80% at the tested concentrations. Of note, this reduction was observed under both baseline (non-arrhythmic) as well as pro-arrhythmic conditions. Together this suggests, that treatment with anti-arrhythmic agents induce an anti-arrhythmic cell population, that reduces the cells' susceptibility to arrhythmias. This anti-arrhythmic cells population makes the obtained cell population highly attractive for cardiac cell therapies mitigating the risk of previously reported arrhythmias following cell transplantation.

Furthermore, our data suggest that the changes in cardiomyocyte function related to the cells' electrophysiological properties being associated with the sustained changes in gene expression including CACNA1G, GJA5, NPPA and/or NPPB. Importantly, the modified properties of the stem-cell derived cardiomyocytes are induced (directly or indirectly) on a gene expression level by exposure to anti-arrhythmic agents and not necessarily due to the common mechanism of action related to direct effects by modulation of ion-channel activity.

### Example 4

In order to test whether combinations of antiarrhythmic agents can further reduce the arrhythmic potential of stem-cell derived cardiomyocytes, we subjected the stem-cell derived cardiomyocytes to the same assay of Ca²⁺-recordings as described in Example 3 and applied various combinations of class I, II and/or III antiarrhythmic agents and compared the beat-to-beat variability to single compound treatments.

### Experimental Procedure

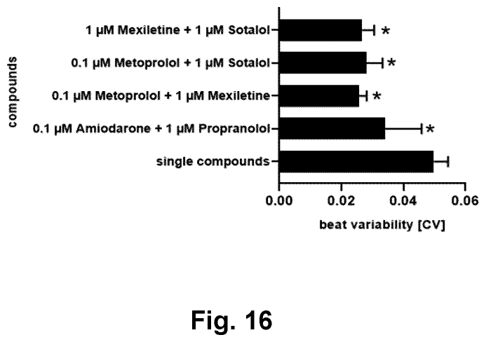

Induced pluripotent stem cell derived cardiomyocytes were treated with combinations of anti-arrhythmic agents for 72 h followed by a 24 h recovery phase before measurement. The agents were applied at the following concentrations: 1 μM sotalol, 0.1 μM amiodarone, 0.1 μM metoprolol and 1 μM mexiletine. All measurements were conducted under proarrhythmic conditions after moxifloxacin treatment. The results show that combination of class III agents with either class I and/or class II are more efficient in reducing the pro-arrhythmic potential than single agents alone with a reduction of 46.4% or 31.4% and 46.4%, respectively (FIG. 16). Similarly, the combination of class I and II showed a reduction by 48.3% compared to single compound treatment.

These results show that combining anti-arrhythmic agents results in additional reduction of the beat-to-beat variability. Consequently, the obtained cardiomyocyte cell population has a further reduced pro-arrhythmic potential compared to single agents alone and thus further mitigates the risk of inducing arrythmias in vivo.

Overall, the results of above examples show that exposure of stem cell-derived cardiomyocytes to anti-arrhythmic drugs induces a profound and sustained reduction in pro-arrhythmic potential thereby obtaining a cell population with antiarrhythmic properties.

The antiarrythmic cardiomyocyte cell population represents a superior cell source for transplantation by reducing the risk of graft-induced arrhythmias and/or tachycardias.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for obtaining an antiarrythmic cardiomyocyte cell population comprising the step of culturing stem cell derived cardiomyocytes in a medium comprising one or more anti-arrhythmic agents.

2. The method according to claim 1, wherein the antiarrhythmic agent is selected from the list of class I, class II, and class III antiarrhythmic agents or combinations thereof.

3. The method according to claim 2, wherein the class I antiarrhythmic agent is lidocaine or mexiletine, the class II antiarrhythmic agent is metoprolol or propranolol, and/or the class III antiarrhythmic agent is amiodarone or sotalol, or a combination thereof.

4. The method according to claim 2, wherein the antiarrhythmic agent is selected from the list of class III in combination with class I and/or class II antiarrhythmic agents.

5. The method according to claim 4, wherein the antiarrhythmic agent is lidocaine and amiodarone, mexiletine and sotalol, metoprolol and sotalol, amiodarone and propranolol, lidocaine and sotalol, amiodarone and metoprolol, amiodarone and mexiletine and/or sotalol and propranolol.

6. (canceled)

7. A method of treating heart failure comprising administering an antiarrythmic cardiomyocyte cell population to a subject in need thereof.

8. The method according to claim 7, wherein the antiarrhythmic cardiomyocyte cell population has at least 50% reduction in coefficient of variation (CV) or beat to beat variability when compared to stem cell derived cardiomyocytes.

9. The method according to claim 7, wherein the antiarrhythmic cardiomyocyte cell population has regulation of expression of gene selected from the list of GJA5, CACNA1G, NPPA and NPPB.

10. The method according to claim 9, wherein the antiarrhythmic cardiomyocyte cell population has upregulation of expression of GJA5 and/or CACNA1G and downregulation NPPA and/or NPPB.

11. The method according to claim 10, wherein the antiarrhythmic cardiomyocyte cell population has an at least 1.5 times upregulation of GJA5, an at least 2 times upregulation of CACNA1G, an at least 2 times downregulation of NPPA and/or an at least 4 times downregulation of NPPB when compared to stem cell-derived cardiomyocytes.

12. A kit comprising an antiarrhythmic agent and stem cell-derived cardiomyocytes.

13. (canceled)

14. (canceled)

15. A composition comprising stem cell derived cardiomyocytes, one or more antiarrythmic agents and optionally a biomaterial.

16. A method of treating heart failure comprising the step of administering the composition of claim 15 to a subject in need thereof.

17. The kit according to claim 12, wherein the antiarrhythmic agent is selected from the list of class I, class II, and class III antiarrhythmic agents or combinations thereof.

18. The kit according to claim 17, wherein the class I antiarrhythmic agent is lidocaine, or mexiletine, the class II antiarrhythmic agent is metoprolol or propranolol, and/or the class III antiarrhythmic agent is amiodarone or sotalol, or a combination thereof.

19. A method of treating heart failure comprising administering an antiarrythmic cardiomyocyte cell population to a subject in need thereof, wherein said antiarrythmic cardiomyocyte cell population is obtained by the method of claim 1.

20. The method according to claim 19, wherein the antiarrhythmic cardiomyocyte cell population has at least 50% reduction in coefficient of variation (CV) or beat to beat variability when compared to stem cell derived cardiomyocytes.

21. The method according to claim 19, wherein the antiarrhythmic cardiomyocyte cell population has regulation of expression of gene selected from the list of GJA5, CACNA1G, NPPA and NPPB.

22. The method according to claim 21, wherein the antiarrhythmic cardiomyocyte cell population has upregulation of expression of GJA5 and/or CACNA1G and downregulation NPPA and/or NPPB.

23. The method according to claim 22, wherein the antiarrhythmic cardiomyocyte cell population has an at least 1.5 times upregulation of GJA5, an at least 2 times upregulation of CACNA1G, an at least 2 times downregulation of NPPA and/or an at least 4 times downregulation of NPPB when compared to stem cell-derived cardiomyocytes.
